(19) United States
(12) Patent Application Publication
(10) Pub. No.: US 2023/0165821 A1
(43) Pub. Date: Jun. 1, 2023

(54) ELAFIBRANOR FOR THE TREATMENT OF PRIMARY SCLEROSING CHOLANGITIS

(71) Applicant: Genfit, Loos (FR)

(72) Inventor: Alice Roudot, Lomme (FR)

(21) Appl. No.: 17/920,260

(22) PCT Filed: May 18, 2021
(86) PCT No.: PCT/EP2021/063075

§ 371 Date: Oct. 20, 2022

(30) Foreign Application Priority Data
May 18, 2020 (EP) 20305513.2

(51) Int. Cl. A61K 31/192; A61K 9/20; A61P 1/16

(52) U.S. Cl. CPC A61K 31/192; A61K 9/20; A61P 1/16

(57) ABSTRACT

The present invention relates to elafibranor or its active metabolite for use in a method for treating primary sclerosing cholangitis.

### TECHNICAL FIELD

The present invention relates to the field of medicine, in particular to the treatment of primary sclerosing cholangitis.

### BACKGROUND

Bile is a digestive liquid that is made in the liver. It travels through the bile ducts to the gallbladder and the small intestine, where it helps digest fats and fatty vitamins.

Cholestasis is a condition that results from an impairment of bile formation or bile flow to the gallbladder and duodenum (first section of the small intestine). The effects of cholestasis are profound and widespread, leading to worsening liver disease and systemic illness, liver failure, and the need for liver transplantation. Cholestasis may be classified as intrahepatic or extrahepatic. Intrahepatic cholestasis primarily involves the bile canaliculi and the intrahepatic bile ducts. Extrahepatic cholestasis involves the extrahepatic ducts, the common hepatic duct or the common bile duct.

Primary Sclerosing Cholangitis (PSC) is a chronic, or long-term, disease that slowly damages the extra- and intrahepatic bile ducts. In patients with PSC, the bile ducts become blocked due to inflammation and deteriorate. This causes bile to accumulate in the liver, where it gradually damages liver cells and causes cirrhosis, or damage of the liver.

Many people with PSC will ultimately need a liver transplant, typically about 10 years after being diagnosed with the disease. PSC may also lead to bile duct cancer.

There is currently no therapy that significantly reduces the risk of death or the need for liver transplantation, which still remains the only solution for patient survival.

### SUMMARY OF INVENTION

A clinical study has surprisingly shown that the treatment of PSC can be achieved with an oral daily dose of elafibranor (2-(2,6-dimethyl-4-{3-[4-(methylsulfanyl)phenyl]-3-oxopropen-1-yl}phenoxy)-2-methylpropanoic acid) comprised between 30 and 70 mg/day.

Therefore, according to a first aspect, the present invention relates to dosage forms suitable for oral administration of 30 to 70 mg/day of a compound selected from elafibranor (ELA) and its active metabolite GFT1007, or of a pharmaceutically acceptable salt thereof, in particular of 40 to 60 mg/day.

The invention more particularly relates to a pharmaceutical composition suitable for oral administration of a daily dose of between 30 and 70 mg of a compound selected from elafibranor and 2-[2,6-dimethyl-4-[3-[4-(methylthio)phenyl]-3-oxo-propyl]phenoxy]-2-methylpropanoic acid, or of a pharmaceutically acceptable salt of said compound, wherein said pharmaceutical composition is a unit dosage form.

In a particular embodiment, the invention relates to a pharmaceutical composition comprising between 30 and 70 mg of elafibranor or of GFT1007, wherein said pharmaceutical composition is a unit dosage form suitable for oral administration. In another embodiment, the pharmaceutical composition comprises between 40 and 60 mg of elafibranor or of GFT1007. In yet another embodiment, the pharmaceutical composition comprises about 40 mg of elafibranor or of GFT1007, such as 35, 36, 37, 38, 39, 40, 41, 42, 43, 44 or 45 mg of elafibranor or of GFT1007. In a particular embodiment, the pharmaceutical composition comprises 40 mg of elafibranor or of GFT1007.

In a further embodiment, said unit dosage form is selected from solid dosage forms and liquid dosage forms, the unit dosage form more particularly being a pill, a tablet, or a capsule, such as a hard gelatin capsule. In a particular embodiment, the pharmaceutical composition is a tablet. In yet another particular embodiment, the pharmaceutical composition is a tablet comprising 40 mg of elafibranor or of GFT1007.

According to another aspect, the invention relates to a compound selected from elafibranor and 2-[2,6-dimethyl-4-[3-[4-(methylthio)phenyl]-3-oxo-propyl]phenoxy]-2-methylpropanoic acid, or a pharmaceutically acceptable salt thereof, in particular elafibranor, for use in a method for the treatment of primary sclerosing cholangitis (PSC), said method comprising orally administering elafibranor or GFT1007 at a dose comprised between 30 mg/day and 70 mg/day. In yet another aspect, the invention relates to a method for the treatment of a subject having PSC, wherein said method comprises administering to said subject a dose of elafibranor or of GFT1007 comprised between 30 and 70 mg/day.

In a particular embodiment, the method comprises the administration of the pharmaceutical composition of the first aspect. In another particular embodiment, the method comprises the oral administration of a single unit dosage form comprising the dose of the compound, in particular elafibranor, to be administered, or of several unit dosage forms comprising a fraction of the daily dose to be administered. In a particular embodiment, the method comprises administering said compound, in particular elafibranor or a pharmaceutically acceptable salt thereof, more particularly elafibranor, at a dose of 40 mg/day. In a specific embodiment, the method comprises the oral administration of a tablet comprising 40 mg of elafibranor or of GFT1007, once daily.

### DETAILED DESCRIPTION OF THE INVENTION

The term “treatment” or “treating” refers to any act intended to ameliorate the health status of a subject, such as therapy, prevention, prophylaxis or delayed progression of a disease in a subject in need thereof. The treatment involves the administration of elafibranor to a subject having a declared disease to prevent, cure, delay, reverse, or slow down the progression of the disease, thereby improving the condition of the subject. A treatment may be also administered to subjects that are either healthy or at risk of developing PSC.

The term “subject” refers to a mammal, preferably to a human. The subjects to be treated according to the invention can be appropriately selected on the basis of several criteria associated with PSC pathological processes such as previous and/or present drug treatments, associated pathologies, genotype, exposure to risk factors, as well as any other relevant biomarker that can be evaluated by means of any suitable immunological, biochemical, or enzymatic method.

Illustrative methods to synthesize elafibranor include those described in PCT applications WO2004/005233 and WO2005/005369.

In some embodiments of the invention, GFT1007, the active metabolite of elafibranor, is used. GFT1007 is 2-[2,6-dimethyl-4-[3-[4-(methylthio)phenyl]-3-oxo-propyl]phenoxy]-2-methylpropanoic acid. Its properties and synthesis were described in PCT application WO2007/147879, where it is referred to as compound 1.

According to the present invention, the pharmaceutical composition of the invention may include a stereoisomer of elafibranor or of GFT1007 or a salt of elafibranor or of GFT1007.

A stereoisomer is an isomeric compound that has the same molecular formula and sequence of bonded atoms, but differs in the 3D-dimensional orientations of its atoms in space. The stereoisomers include enantiomers, diastereoisomers, cis-trans and E-Z isomers, conformers and tautomers.

“Pharmaceutically acceptable salts” include inorganic as well as organic acids salts. Counterions may be selected from the following the non-exhaustive list : ammonia, L-arginine, benethamine, benzathine, tert-butylamine (erbumine), calcium hydroxide, choline hydroxide, deanol, diethanolamine (2,2′-iminobis(ethanol), diethylamine, epolamine (1-(2-hydroxyethyl)pyrrolidine), 2-(diethylamino)-ethanol, ethanolamine (2-aminoethanol), ethylenediamine, glycine, hydrabamine, 1H-imidazole, L-Lysine, magnesium hydroxide, meglumine (N-methyl-glucamine), 4-(2-hydroxyethyl)-morpholine, piperazine, potassium hydroxide, sodium hydroxide, triethanolamine (2,2′,2″-nitrilo-tris(ethanol)), tromethamine, zinc hydroxide, in particular tromethamine, potassium, sodium, benethamine, benzathine, L-arginine, ethanolamine, meglumine, glycine, erbumine, L-lysine, epolamine, choline, preferably tromethamine, potassium, sodium, benethamine, benzathine, L-arginine, more preferably tromethamine, potassium, sodium, L-arginine, more particularly tromethamine. In particular embodiments, the invention relates to an ammonia, L-arginine, benethamine, benzathine, tert-butylamine (erbumine), calcium, choline, deanol, diethanolamine (2,2′-iminobis(ethanol), diethylamine, epolamine (1-(2-hydroxyethyl)pyrrolidine), 2-(diethylamino)-ethanol, ethanolamine (2-aminoethanol), ethylenediamine, glycine, hydrabamine, 1H-imidazole, L-Lysine, magnesium, meglumine (N-methyl-glucamine), 4-(2-hydroxyethyl)-morpholine, piperazine, potassium, sodium, triethanolamine (2,2′,2″-nitrilo-tris(ethanol)), tromethamine or zinc salt of elafibranor. In a further particular embodiment, the salt of elafibranor is selected from a tromethamine, potassium, sodium, L-arginine, benethamine, benzathine, ethanolamine, meglumine, glycine, erbumine, L-lysine, choline, epolamine, magnesium or 2-amino-2-methyl-propan-1-ol salt of elafibranor.

The pharmaceutical composition of the invention can comprise one or several excipients or vehicles, acceptable within a pharmaceutical context (e.g. saline solutions, physiological solutions, isotonic solutions, etc., compatible with pharmaceutical usage and well-known by one of ordinary skill in the art). These compositions can comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. These compositions can be formulated solid or liquid unit dosage forms for oral administration. In a particular embodiment, the pharmaceutical composition of the invention is a solid dosage form, such as a pill, a tablet, or capsule (e.g. a hard gelatin capsule). In a particular embodiment, the pharmaceutical composition is a tablet or capsule, in particular a tablet or hard gelatin capsule, more particularly a tablet.

In an embodiment, the pharmaceutical composition is suitable for oral administration of between 30 mg/day and 70 mg/day of a compound selected from elafibranor, GFT1007 and a pharmaceutically acceptable salt of elafibranor of of GFT1007 to a subject with PSC.

In another embodiment, the pharmaceutical composition of the invention comprises between 30 and 70 mg of a compound selected from elafibranor, GFT1007 and a pharmaceutically acceptable salt of elafibranor or of GFT1007, wherein said pharmaceutical composition is a unit dosage form suitable to administer between 30 and 70 mg/day of said compound to a subject with PSC.

In another particular embodiment, the pharmaceutical composition of the invention is suitable for administration to a subject with PSC, said pharmaceutical composition comprising between 30 and 70 mg of a compound selected from elafibranor, GFT1007 and a pharmaceutically acceptable salt of elafibranor or of GFT1007, wherein said pharmaceutical composition is a unit dosage form suitable to administer between 30 mg/day and 70 mg/day of said compound to a subject with PSC.

The pharmaceutical composition of the invention is a unit dosage form suitable for oral administration of 30 to 70 mg/day of elafibranor, of GFT1007, of a pharmaceutically acceptable salt of elafibranor or of a pharmaceutically acceptable salt of GFT1007. More particularly, the pharmaceutical composition of the invention is a unit dosage form suitable for oral administration of 30 to 70 mg/day of elafibranor or of GFT1007. For example, in a particular embodiment, a single unit dosage form comprises the daily dose of elafibranor or of GFT1007 to be administered. An illustration of this embodiment includes a unit dosage form of elafibranor or of GFT1007 which comprises 40 mg of elafibranor or of GFT1007, when the daily dose to be administered to the subject is 40 mg/day. In another embodiment, the pharmaceutical composition is a unit dosage form comprising a fraction of the daily dose of elafibranor or of GFT1007. In this embodiment, the pharmaceutical composition can be administered several times a day and/or several units of the pharmaceutical composition can be used concomitantly to achieve the desired daily dose. An illustrative, non-limiting embodiment of such a unit dosage form comprises 10 mg of elafibranor or of GFT1007, meaning that to achieve a 30 to 70 mg/day dose, 3 to 7 unit dosage forms should be administered each day. Representative unit dosage forms, such as tablets or capsules (in particular hard gelatin capsules) useful in the context of the present invention include oral dosage forms comprising 5, 10, 15, 20, 25, 30, 35, 40, 45, 50, 55, 60, 65 or 70 mg of elafibranor or of GFT1007.

According to another aspect, the invention relates to elafibranor or GFT1007 for use in the treatment of primary sclerosing cholangitis (PSC), said method comprising orally administering elafibranor or GFT1007 at a dose comprised between 30 mg/day and 70 mg/day. In yet another aspect, the invention relates to a method for the treatment of a subject having PSC, wherein said method comprises administering to said subject a dose of elafibranor or of GFT1007 comprised between 30 and 70 mg/day.

In a particular embodiment, the method comprises the administration of the pharmaceutical composition of the invention. In another particular embodiment, the method comprises the oral administration of a single unit dosage form comprising the daily dose of elafibranor or of GFT1007 to be administered, or of several unit dosage forms comprising a fraction of the daily dose to be administered. In a specific embodiment, the method comprises the oral administration of a tablet comprising 40 mg or 60 mg of elafibranor or of GFT1007, once daily. In a more specific embodiment, the method comprises the oral administration of a tablet comprising 40 mg of elafibranor or of GFT1007, once daily.

In yet another embodiment, elafibranor or GFT1007 is administered in the morning, more particularly in fasting conditions.

In a preferred variant of all the aspects and embodiments disclosed above, the compound in the invention is elafibranor or a pharmaceutically acceptable salt thereof.

The invention is further described with reference to the following, non-limiting, examples.

### EXAMPLES

### Example 1: Clinical Trial on PSC

Patients with clinical PSC have elevated alkaline phosphatase (ALP) and bilirubin levels. Several studies show that ALP can serve as stratifier, and potentially as surrogate endpoint for clinical trials in PSC (De Vries, E.M.G. et al., Alkaline phosphatase at diagnosis of primary sclerosing cholangitis and 1 year later: evaluation of prognostic value. Liver International (2016) pp1478-3223). Some studies underline also elevated levels of bilirubin (Denau, M.R. et al., The Natural History of Primary Sclerosing Cholangitis in 781 Children: A Multicenter, International Collaboration. Hepatology (2017), Vol. 66, N° 2 pp518-527).

This clinical trial has been performed to assess the tolerability, safety and reduction of different parameters, including alkaline phosphatase of once-a-day administrations of oral doses of elafibranor (ELA) in patients suffering from PSC. This was a phase I, single-center, double-blind, placebo-controlled, randomised, ascending multiple-dose study in healthy male volunteers. Doses of 40 mg or 60 mg once-a-day during 14 days were tested.

Thirty 19-40 years old Caucasian male volunteers were included: 9 for each treatment group and 12 for the placebo group.

The compound used was ELA, supplied in hard gelatin capsules dosed at 10 mg. Dose per administration was of 40 mg or 60 mg. Administration was repeated once-a-day, with an oral administration around 8 a.m. with 200 mL tap water, in sitting position, in fasting conditions, from day 1 to day 14. Matching placebo was presented as identical capsules.

TABLE 1

Dosage of Alkaline Phosphatase
Assessment of ALP level

| | | | |
|---|---|---|---|
| Amount of ELA (in mg/day) | 40 | 60 | - |
| Number of patients | 9 | 9 | 12 |
| Measure of ALP Day 15 change from the baseline | -8.3 | -20.7 | -7.6 |

PSC patients treated with both elafibranor doses (40 mg and 60 mg) improved alkaline phosphatase levels compared to placebo group

TABLE 2

Dosage of bilirubin
Assessment of bilirubin level

| | | | |
|---|---|---|---|
| Amount of ELA (in mg/day) | 40 | 60 | - |
| Number of patients | 9 | 9 | 12 |
| Measure of total bilirubin Day 15 change from the baseline | -2.07 | -2.46 | -0.72 |

PSC patients treated with both elafibranor doses (40 mg and 60 mg) improved total bilirubin levels compared to placebo group

No serious adverse events were reported. A good clinical tolerance was observed for repeated administration of ELA administered at either doses in healthy volunteers.

The results are summarized in tables 1 and 2.

Alkaline phosphatase level was assessed at baseline (day 1) and day 15. The results show that both doses of elafibranor decreased mean ALP compared with placebo (table 1). Furthermore, PSC patients treated with both elafibranor doses improved total bilirubin levels compared to placebo group (table 2).

Previous and ongoing clinical trials assessing the efficacy of elafibranor on different diseases, in particular on non-alcoholic steatohepatitis or primary biliary cholangitis, were conducted with a daily dose of 80 mg or 120 mg. It is surprisingly shown herein that elafibranor may be used at a lower dose to treat PSC.

1. A pharmaceutical composition suitable for oral administration of a daily dose of between 30 and 70 mg of a compound selected from elafibranor and 2-[2,6-dimethyl-4-[3-[4-(methylthio)phenyl]-3-oxo-propyl]phenoxy]-2-methylpropanoic acid, or of a pharmaceutically acceptable salt of said compound, wherein said pharmaceutical composition is a unit dosage form.

2. The pharmaceutical composition according to claim 1, suitable to administer between 40 and 60 mg of said compound.

3. The pharmaceutical composition according to claim 1, suitable to administer 40 mg of said compound.

4. The pharmaceutical composition according to claim 1, wherein said unit dosage form is selected from solid dosage forms and liquid dosage forms.

5. The pharmaceutical composition according to claim 1, wherein said unit dosage form is a tablet.

6. The pharmaceutical composition according to claim 1, wherein said compound is elafibranor or a pharmaceutically acceptable salt thereof.

7. A pharmaceutical composition comprising elafibranor, for administration of a daily dose of 40 mg of elafibranor to a subject in need thereof.

8. A method for treating primary sclerosing cholangitis (PSC) comprising orally administering a compound selected from elafibranor and 2-[2,6-dimethyl-4-[3-[4-(methylthio)phenyl]-3-oxo-propyl]phenoxy]-2-methylpropanoic acid, or a pharmaceutically acceptable salt thereof, wherein said compound is administered at a dose of between 30 mg/day and 70 mg/day.

9. The method according to claim 8, said method comprising administering the pharmaceutical composition according to claim 1.

10. The method according to claim 8, wherein said compound is administered at a dose of 40 mg/day.

11. The method according to claim 10, wherein said compound is elafibranor or a pharmaceutically acceptable salt thereof.

12. The method according to claim 10 wherein said compound is elafibranor.

13. The method according to claim 8 said method comprising administering a pharmaceutical composition comprising between 30 and 70 mg of said compound, wherein said pharmaceutical composition is a unit dosage form suitable for oral administration.

14. The method according to claim 13, wherein the pharmaceutical composition comprises between 40 and 60 mg of said compound.

15. The method according to claim 13 wherein the pharmaceutical composition comprises 40 mg of said compound.

16. The method according to claim 13, wherein said unit dosage form is selected from solid dosage forms and liquid dosage forms.

17. The method compound according to claim 13, wherein said unit dosage form is a tablet.

18. The method according to claim 13, wherein said method comprises the oral administration of a tablet comprising 40 mg of said compound once daily.

19. The method according to claim 13, wherein said compound is elafibranor or a pharmaceutically acceptable salt thereof.

20. The method according to claim 13, wherein said compound is elafibranor.
